(19) United States
(12) Patent Application Publication
(10) Pub. No.: US 2023/0240310 A1
(43) Pub. Date: Aug. 3, 2023

(54) PRODUCTION OF DAIRY AND DAIRY ANOLOGUE PRODUCTS WITH PICHIA KLUYVERI YEAST

(71) Applicant: Chr. Hansen A/S, Hoersholm (DK)

(72) Inventors: Patrizia Buldo, Hoersholm (DK); Raquel Fernandez, Hoersholm (DK); Kasui Tang, Arpajon (FR); Kristian Fog Nielsen, Hoersholm (DK); Asger Geppel, Hoersholm (DK)

(73) Assignee: Chr. Hansen A/S, Hoersholm (DK)

(21) Appl. No.: 18/011,978

(22) PCT Filed: Jun. 30, 2021
(86) PCT No.: PCT/EP2021/067971

§ 371 Date: Dec. 21, 2022

(30) Foreign Application Priority Data
Jun. 30, 2020 (EP) 20183014.8
Sep. 29, 2020 (EP) 20198931.6

(51) Int. Cl. A23C 9/12; A23C 11/10; A23L 11/50; A23L 11/65; A23L 25/00

(52) U.S. Cl. CPC A23C 9/12; A23C 11/106; A23L 11/50; A23L 11/65; A23L 25/40; A23Y 2220/67; A23Y 2240/75; A23Y 2280/15

(57) ABSTRACT

The invention relates to preparation of fermented food products, including dairy or dairy analogue products, with Pichia kluyveri and lactic acid bacteria in the presence of carbohydrate(s) which is sucrose, fructose, glucose or mixtures thereof. Fermentation of such products can be made using a starter culture composition comprising Pichia kluyveri strain(s) and lactic acid bacteria strain(s). The composition optionally comprises carbohydrate(s) which is sucrose, fructose and/or glucose.

### FIELD OF THE INVENTION

The present invention relates to the field of food technology, in particular to the preparations of dairy products or dairy analogue products by fermentation.

### BACKGROUND OF THE INVENTION

Dairy products are products that use the milk of milking animal (e.g., cow, buffalo, goat, sheep) as starting material. They can be obtained by fermenting the milk, with or without compositional modification, with suitable microorganisms and resulting in reduction of pH. Dairy analogues are food products designed as an alternative to traditional animal-derived foodstuff. The most commonly known examples of dairy analogues are cheese analogues (imitation cheeses, cheese-like products, cheese spreads), fermented dairy analogues (yoghurt-like products), butter analogues, and dairy dessert analogues. Such products are becoming popular in recent years and used by an increasing number of consumers for medical reasons (e.g., lactose intolerance, cow's milk allergy), lifestyle choice (e.g., vegetarians, flexitarian, religious groups) or sustainability. Furthermore, there is a gradual shift from overdependence on animal fat and proteins because of health implications. It is considered that consumption of fats and proteins of plant origin are associated with a reduced risk of cardiovascular and degenerative diseases.

There is an ongoing demand to provide fermented dairy analogue products with improved taste, aroma and texture. It is preferred that there is very little formation of unwanted byproducts in the manufacturing process, such as alcohol. An alcohol content of lower than 0.5% (wt), in particular lower than 0.1% (wt), is desirable.

Dairy analogues prepared from legumes have been consumed for centuries in Asia and are becoming popular in the Western world. Food legumes represent a diverse group of plants. They are found worldwide and consumed in practically every country of the world. About 20% of the protein currently available in developing countries is derived from food legumes.

Legume products tend to have beany and painty off-flavors. Fermentation may render the raw material more palatable, with desirable changes in the texture and organoleptic characteristics (flavor, aroma, appearance and consistency). Some attempts have been made to ferment legume products to remove beany flavors, for example with lactic acid bacteria (Mita) et al. “Fermentation of soy milk by lactic acid bacteria. A review.” Journal of food protection 42.11 (1979): 895-899).

It is known that the rate of acid development is a critical factor in fermentation of food products. A rapid acidification of the raw material prevents growth of undesirable microorganisms and is also important for aroma, texture and flavor of the end-product. However, in the case of soybean, some researchers observed that soymilk fermentation with probiotic bacteria may take longer time to complete and produces undesirable changes not acceptable to the consumer (Donkor, Osaana N., et al. “Rheological properties and sensory characteristics of set-type soy yogurt.” Journal of Agricultural and Food Chemistry 55.24 (2007): 9868-9876). Often, commercial products incorporates flavoring agents to mask off-flavors. Industrially, short fermentation times are preferred to increase output and efficiency. Therefore, there is a need to provide fermented food products with short fermentation time and/or improved taste, aroma and texture. Furthermore, in view of the global shift to plant-based diets, there is a need to provide fermented dairy analogues products from plant material with improved taste, aroma and texture.

### SUMMARY OF THE INVENTION

The present invention provides methods of producing fermented dairy products (from animal milk) as well as fermented dairy analogue products (from plant material, i.e. plant milk, for example from legumes, nuts or cereals) using the yeast Pichia kluyveri. It has been discovered that the use of Pichia kluyveri in the presence of lactic acid bacteria and certain carbohydrates leads to improved flavor profile and palatability in dairy and dairy analogue products.

By including lactic acid bacteria and providing sucrose, fructose and/or glucose as substrate, a synergistic effect in improving aroma and reducing alcohol formation can be observed.

The aroma of dairy products is comprised of a vast array of volatile organic compounds (VOCs), such as alcohols, aldehydes, esters, free fatty acids, ketones, lactones, phenolic compounds and sulphur compounds (Urbach, G. (1993). Relations between cheese flavor and chemical composition. International Dairy Journal, 3, 389-422). Esters contributed to the flavor is concentration dependent. At low concentrations, esters contribute positively to the overall flavor balance; at high concentrations, they may cause a fruity flavor defect (S.-Q. Liu, R. Holland, V. L. Crow (2004) Review: Esters and their biosynthesis in fermented dairy products: a review. International Dairy Journal 14, 923-945). Esters are important contributors to the flavor, and they used to be described with the term of “fruity”, such as apple, banana, pear, pineapple, etc.

Surprisingly, it was found that specific combinations of features resulted in an increased generation of VOCs, such as the total amount of esters, with a favorable aroma profile.

In a first aspect, the present invention provides the use of lactic acid bacteria and carbohydrate(s) selected from the group consisting of sucrose, fructose and glucose for increasing aroma and/or reducing alcohol in a food product fermented by Pichia kluyveri. Such food products may be dairy products and dairy analogues. Pichia kluyveri, lactic acid bacteria and one or more carbohydrates selected from the group consisting of sucrose, fructose and glucose can be used together for fermenting a dairy product or dairy analogue product from suitable materials such as animal milk or plant milk.

The carbohydrates, sucrose, fructose and/or glucose are provided in the starting material prior to, at the start, or during fermentation. The compounds can be added directly or provided indirectly via microorganisms. In this, fructose or glucose can be provided by adding suitable sugars for the lactic acid bacteria, which are able to metabolize the sugars and release fructose or glucose during fermentation. Sucrose is a disaccharide consisting of one glucose and one fructose molecule. Depending on the preference of the bacteria, glucose or fructose is utilize and the less preferred monosaccharide can be released.

More preferably, the carbohydrate is sucrose or fructose.

Furthermore, lactic acid can also be added to enhance the growth of Pichia kluyveri and/or reduce alcohol level.

In a further aspect, the present invention provides methods of producing fermented food product, such as dairy products and dairy analogue products, comprising fermenting suitable substrates with Pichia kluyveri, in the presence of one or more of lactic acid bacteria and one or more carbohydrates selected from the group consisting of sucrose, fructose and glucose.

Food products prepared according to the present invention has an increased viscosity as result of the fermentation. Viscosity of the product can be measured by a flow test measuring the shear stress of the product as function of shear rate.

In preferred embodiments, the viscosity of the fermented food products according to the present invention is higher than 5 Pa, such as higher than 10 Pa, such as higher than 20 Pa, such as higher than 30 Pa, such as higher than 40 pa, such as higher than 50 Pa, such as higher than 60 Pa, such as higher than 70 Pa, such as higher than 80 Pa, such as higher than 90 Pa, such as higher than 100 Pa (measured at 13° C. at a shear rate of 300 1/s after 7 days of storage).

Dairy Analogue Products

For dairy analogue products, a plant-based material can be used as starting material. Such material is of plant origin and comprises substantially plant materials but has been selected because the end result is dairy-like and can be used to replace dairy products. Dairy analogue products (or simply “dairy analogues”) refer to dairy-like products, which are products used as culinary replacements for dairy products, prepared where one or more animal milk constituents have been replaced with other ingredients and the resulting food resembles the original product. Examples include analogues of milk, cream, cheese, yogurt, spread, butter, ice cream and the like.

In a further aspect, the present invention provides methods of producing fermented dairy analogue product, comprising providing a plant-based material (for example plant milk comprising legumes, cereals, nuts) and fermenting the material with Pichia kluyveri and lactic acid bacteria in the presence of one or more carbohydrates selected from the group consisting of sucrose, fructose and glucose.

Provided herein are methods of producing fermented dairy analogue products from plant milk base, comprising providing milk base as substrate, adding at least one Pichia kluyveri strain and at least one lactic acid bacteria strain to the substrate, fermenting the substrate with Pichia kluyveri in the presence of lactic acid and sucrose.

Provided herein are methods of producing fermented dairy analogue products from plant milk base, comprising providing milk base as substrate, adding at least one Pichia kluyveri strain and at least one lactic acid bacteria strain to the substrate, fermenting the substrate with Pichia kluyveri in the presence of lactic acid and fructose.

Provided herein are methods of producing fermented dairy analogue products from plant milk base, comprising providing milk base as substrate, adding at least one Pichia kluyveri strain and at least one lactic acid bacteria strain to the substrate, fermenting the substrate with Pichia kluyveri in the presence of lactic acid and glucose.

“Plant milk” is a general term for products derived from a plant source that resembles milk (sometimes also called imitation milk). The similarity of the functional properties, nutritive value, the presence of fat and protein and sensory characteristics of these milk analogues allow them to be used as substitutes for animal milks. In general, plant milk is manufactured by extracting the plant material in water, filtering off the aqueous extract followed by homogenization and heat treatment in order to improve the suspension and microbial stability of the product.

Dairy Products

In another aspect, the present invention provides methods of producing fermented dairy products from animal milk, comprising providing milk base as substrate, adding at least one Pichia kluyveri strain and at least one lactic acid bacteria strain to the substrate, fermenting the substrate with Pichia kluyveri in the presence of lactic acid and sucrose.

In another aspect, the present invention provides methods of producing fermented dairy products from animal milk, comprising providing milk base as substrate, adding at least one Pichia kluyveri strain and at least one lactic acid bacteria strain to the substrate, fermenting the substrate with Pichia kluyveri in the presence of lactic acid and fructose.

In another aspect, the present invention provides methods of producing fermented dairy products from animal milk, comprising providing milk base as substrate, adding at least one Pichia kluyveri strain and at least one lactic acid bacteria strain to the substrate, fermenting the substrate with Pichia kluyveri in the presence of lactic acid and glucose.

Such dairy products may be kefir. Kefir is a fermented milk drink that originated in the Caucasus Mountains made with kefir “grains”, a yeast/bacterial fermentation starter. Traditionally, it is prepared by inoculating cow, goat or sheep milk with kefir grains and fermented at ambient temperatures, generally overnight. Fermentation of the lactose yields a sour, carbonated, slightly alcoholic beverage, with a consistency and taste similar to thin yogurt. Carbonate and alcohol are mainly produced by yeasts. Today, kefir is capturing new markets with more than 100 innovations a year and companies needs modern solutions to produce kefir in a standard process without using grains and with very small amount of gas to avoid blowing in the bottles. However, in some countries the yeast is needed for legal denomination.

It has been discovered that by Pichia kluyveri can be advantageously used to prepare dairy products with good organoleptic properties and high cell counts that can meet the legal requirement (for example >10⁴ cfu/g for kefir products according to CODEX STAN 243-2003) during the shelf life. The effect can be seen for such products stored at cold temperature (<7° C.) and analyzed at week 2, 3, 4 or 5. Particularly when fructose, glucose or sucrose were used in the fermentation base, high yeast cell count can be maintained compared with other types of sugars.

Starter Culture Composition

The present invention also provides a starter culture composition comprising a Pichia kluyveri and at least one lactic acid bacteria strain. The strain may be one of Lactobacillus spp., Bifidobacterium spp., Streptococcus spp., Lactococcus spp., Pedicoccus spp., Leuconostoc spp., Oenococcus spp.

The starter culture composition may optionally comprise one or more carbohydrates selected from the group consisting of sucrose, fructose and glucose.

The synergism of the microorganisms can contribute positively to the flavor profile of fermented food products as well as the alcohol content. Improvements can be observed when compared to using Pichia kluyveri alone.

In other aspects, the present invention also provides fermented food products, for example dairy products and dairy analogue products. Products described herein comprise Pichia kluyveri and lactic acid bacteria, including the Pichia kluyveri strains 1, 2, and 3 described in the present application. Such products can be obtained by the methods described in the application.

### Definitions

Prior to outlining the present invention in more details, a set of terms and conventions is first defined:

Unless specifically stated, the term “milk” is used broadly and encompass both animal milk and plant milk.

Milk bases may include, but are not limited to, solutions or suspensions of any milk or milk like products comprising protein, such as whole or low fat milk, skim milk, buttermilk, reconstituted milk powder, condensed milk, dried milk.

The term “dairy analogue” as used herein is meant to refers to dairy-like products, which are products used as culinary replacements for dairy products, prepared where one or more milk constituents have been replaced with other ingredients and the resulting food resembles the original product. The milk constituents are replaced completely or substantially with plant material, for example, using planted-based milks derived from legumes (such as soybeans), nuts (such as almonds cashews, coconuts), cereals such as (oat, rice, corn, or wheat). Such plant-based milk, prepared from plant material, is referred to herein as “plant milk” or “plant milk base”.

It should be noted that “dairy analogue”, “plant milk” or “plant milk base” used herein does not refer to alcoholic beverages, or fruit and vegetable juices in general, since such beverages are generally not considered as culinary replacements for dairy products.

Legume

The term “legume” refers to any plant belonging to the family Fabaceae. Fabaceae is a large and economically important family of flowering plants, which is commonly known as the legume family, pea family, bean family or pulse family. A variety of different legumes can be consumed. Legumes typically have a pod or hull that opens along two sutures when the seeds of the legume are ripe. The Fabaceae family includes over 750 genera and 16,000 to 19,000 species.

Examples of “legumes” include peanuts (Arachis hypogaea), pigeon peas (Cajanus cajan), chickpea (Cicer arietinum), soy bean (Glycine max), lentils (Lens culinaris), lupins (Lupinus spp.), peas (Pisum sativum), field peas (Pisum arvense), beans (Phaseolus spp.), common beans (Phaseolus vulgaris) and its various cultivars and varieties, vetches (Vicia spp.), fava beans (Vicia faba), beans (Vigna spp.), cow peas (Vigna unguiculata), azuki beans (Vigna angularis) and bambara beans (Voandzeia subterranea).

Nut

The term “nuts” as used herein can be true nuts from tree or shrubs or culinary nuts which may be drupaceous nuts or seeds that are nut-like. In botanical terms, a nut is a dry one-seeded fruit which is indehiscent (i.e., it does not split open along a definite seam at maturity). Examples of true nuts include acorn (Quercus spp., Lithocarpus spp., and Cyclobalanopsis spp.), chestnut (Castanea spp.), hazelnut (Corylus spp.) and beech nuts (Fagus spp.).

Culinary nuts are those that are not botanically qualified as nuts, but that have a similar appearance and culinary role. Many culinary nuts are seeds of a drupe, referred herein as drupaceous nuts. A drupe is an indehiscent fruit in which an outer fleshy part surrounds a single shell (the pit or stone) hardened endocarp with a seed inside. Drupaceous nuts are seed of drupes. Examples of culinary nuts include drupe seeds such as cashew (Anacardium occidentale), pistachio (Pistacia vera), almond (Prunus dulcis), walnut (Juglans spp.), hickory nuts (Carya spp.), and coconut (Anacardium occidentale). Other examples culinary nuts include Brazil nuts (Bertholletia excelsa) and macadamia (Macadamia spp.). Some culinary nuts are seeds of gymnosperms, such as pine nuts (Pinus spp.) and gingko nuts (Ginkgo biloba).

Cereal

The term “cereal” refers to both true cereal and pseudocereal. True cereal refers to the seeds of plants of the Poaceae family. Examples of true cereals include oat (Avena sativa), rye (Secale cereale), rice (Oryza spp. such as Oryza sativa), sorghum (Sorghum spp., such as Sorghum bicolor), triticale (Triciale), millet (such as finger millet (Eleusine coracana), foxtail millet (Setaria italica), kodo millet (Paspalum scrobiculatum), proso millet (Panicum miliaceum), barnyard millet (Echinochloa spp.)), fonio (Digitaria exilis), teff (Eragrostis tef), barley (Hordeum vulgare), corn (Zea mays), and wheat (Triticum spp.) (such as common wheat (Triticum aestivum), durum wheat (Triticum durum), club wheat (Triticum compactum), Khorasan wheat (Triticum turanicum) and spelt (Triticum spelta)).

Pseudocereal are seed of plants which do not belong to Poaceae family but are used in much the same way as cereals. Examples of pseudocereals include quinoa (Chenopodium quinoa), buckwheat (Fagopyrum esculentum), amaranth (Amaranthus tricolor), breadnut (Brosimum alicastrum), and acacia seed (Acacia spp.).

Animal Milk

The term “animal milk” should be understood as the lacteal secretion obtained by milking of any mammal, such as cows, sheep, goats, buffaloes or camels. Animal milk base can be obtained from any raw and/or processed animal milk material as well as from reconstituted animal milk powder. Animal milk base prepared from milk or milk components from cows is preferred. In some preferred embodiments, the animal milk is pasteurized milk obtained from cows, sheep, goats, buffaloes or camels.

Milk bases may include, but are not limited to, solutions or suspensions of any milk or milk like products comprising protein, such as whole or low fat milk, skim milk, buttermilk, reconstituted milk powder, condensed milk, dried milk.

Milk base may be a solution or suspension of protein(s) that can be found in milk, such as whey proteins, casein proteins or mixtures thereof. Examples of whey proteins include as beta-lactoglobulin, alpha-lactalbumin or mixtures thereof. Examples of casein proteins include alpha-casein, beta-casein, kappa-casein, or mixtures thereof.

A “substrate” is a material that is fermentable by Pichia kluyveri.

In the context of the present application, the term “lactic acid bacteria” is used to refer to food-grade bacteria producing lactic acid as the major metabolic end-product of carbohydrate fermentation. These bacteria are related by their common metabolic and physiological characteristics and are usually Gram-positive, low-GC, acid tolerant, non-sporulating, non-respiring, rod-shaped bacilli or cocci. Lactic acid bacteria represent a group of microorganisms that are functionally related by their ability to produce lactic acid during fermentation.

The inventor discovered that during the fermentation stage, not only are these bacteria able to form lactic acid which is usable by Pichia kluyveri to maintain growth, they are also contribute to the aroma formation and low ethanol production by Pichia kluyveri.

Lactic acid bacteria useful for making fermented products encompass, but is not limited to, bacteria belonging to the genus of Lactobacillus spp., Bifidobacterium spp., Streptococcus spp., Lactococcus spp., Pedicoccus spp., Leuconostoc spp., Oenococcus spp. Examples include but are not limited to Lactobacillus delbrueckii subsp. bulgaricus, Lactobacillus paracasei, Lactobacillus helveticus, Lactobacillus acidophilus, Lactobacillus fermentum, Lactobacillus rhamnosus, Lactobacillus plantarum Lactobacillus buchneri, Lactobacillus curvatus, Lactobacillus sakei, Bifidobacterium breve, Bifidobacterium animalis, Streptococcus thermophilus, Lactococcus lactis, Oenococcus oeni.

In other embodiments, the lactic acid bacteria strain is Streptococcus thermophilus.

The specific selection of strains in the starter culture will depend on the particular type of fermented product to be manufactured.

The term “alcohol-free” herein refers to a content of less than 0.5% (wt) or 5000 ppm

The term “reducing” in the context of the “alcohol production” or “alcohol formation” refers to a lower ethanol production for the same product prepared using both Pichia kluyveri and lactic acid bacteria compared to using Pichia kluyveri alone.

The term “improving the flavor” of a product means to making the product more palatable. This can be determined for example by sensory assessment known to a skilled person in the art.

The term “yogurt analogue” refers to a product which is a culinary replacement for dairy yogurts. Preparation of typical dairy yoghurt products involves the use of symbiotic cultures of Streptococcus thermophilus and Lactobacillus delbrueckii subsp. bulgaricus. When preparing yogurt analogues according to the present invention, using one or both of these cultures will advantageously release lactic acid and/or carbohydrate(s) useful for Pichia kluyveri.

The term “mutant” should be understood as a strain derived from the Pichia kluyveri of the invention by means of e.g. genetic engineering, radiation and/or chemical treatment. It is preferred that the mutant is a functionally equivalent mutant, e.g. a mutant that has substantially the same, or improved, properties as the mother strain. In the present context a mutant of the invention is preferable a mutant with same or improved properties with respect to aroma formation. Such a mutant is a part of the present invention. A mutant may be a strain obtained by subjecting a strain of the invention to any conventionally used mutagenization treatment, including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 1000, no more than 100, no more than 20, no more than 10, or no more than 5, treatments are carried out. In a presently preferred mutant, less than 5%, or less than 1% or even less than 0.1% of the nucleotides in the bacterial genome have been changed (such as by replacement, insertion, deletion or a combination thereof) compared to the mother strain.

### BRIEF DESCRIPTION OF THE FIGURES

FIG. 1 shows fermentation profiles of soy samples fermented with Pichia kluyveri at 30° C.

FIG. 2 shows Pichia kluyveri cell count (cfu/g) in soy samples fermented with Pichia kluyveri after 1 day of storage at 6° C.

FIG. 3 shows the level of ethanol (ppm) in soy samples fermented with Pichia kluyveri after 1 day of storage at 6° C.

FIG. 4 shows Pichia kluyveri cell count (cfu/g) in soy samples fermented with Pichia kluyveri after 1 day of storage at 6° C.

FIG. 5 shows a spider plot from descriptive sensory evaluation (*=Statistical significant attributes) in soy samples fermented with Pichia kluyveri and lactic acid bacteria.

FIG. 6 shows the level of propyl acetate (S/N) present in the soy samples fermented with Pichia kluyveri and lactic acid bacteria.

FIG. 7 shows fermentation time (h) for coconut, soya and oat samples fermented with Pichia kluyveri or Debaryomyces hansenii.

FIG. 8 shows the level of ethanol (ppm) in coconut, soya and oat samples fermented with Pichia kluyveri or Debaryomyces hansenii.

FIG. 9 shows the level of ethanol (ppm) in coconut, soya and oat samples fermented with Pichia kluyveri or Debaryomyces hansenii.

FIG. 10 shows the level of ethanol (ppm) in coconut, soya and oat samples fermented with Pichia kluyveri or Debaryomyces hansenii.

FIG. 11 shows Pichia kluyveri and Debaryomyces hansenii cell count (cfu/g) in kefir samples fermented with the LAB starter culture and the respective yeasts. The samples were stored at 6° C. cell count measured at day 0, day 1, day 7, day 15 and day 28.

### DETAILED DESCRIPTION OF THE INVENTION

The present invention concerns the fermentation of food products by yeasts. Yeasts are eukaryotic microorganisms that inhabit a variety of ecological niches such as water, soil, air and the surface of plants and fruits. Commonly, they are present during the decomposition of ripen fruits and participate in the fermentation process. In this natural environment, the yeasts find nutrients and substrates necessary for their metabolism and fermentative activity.

Yeasts are divided into two large groups namely Saccharomyces and non-Saccharomyces. Regarding their metabolism, yeasts are usually characterized by fermenting a broad spectrum of sugars, among them, glucose, fructose, sucrose, maltose and maltotriose. Saccharomyces cerevisiae is the most studied species and the most utilized in the fermentation of wines and beers due to its excellent fermentative capacity, rapid growth and easy adaptation. Non-Saccharomyces yeasts are a group of microorganisms genetically diverse with specific metabolic characteristics and high potential for using in fermentation processes.

Saccharomyces and non-Saccharomyces yeasts share common pathways for the central metabolism of carbon. Both groups metabolize glucose through glycolysis. However, the mechanisms involved in the regulation of respire-fermentative metabolism can differ significantly among different yeast (Flores et al. “Carbohydrate and energy-yielding metabolism in non-conventional yeasts.” FEMS microbiology reviews 24.4 (2000): 507-529).

Pichia kluyveri, the key fermentation microorganism of the present invention, is a non-saccharomyces yeast which has been used for beer fermentation. The patent WO2014135673 (Chr. Hansen A/S, Denmark) discloses using this yeast to ferment wort and obtain low alcohol beer. As disclosed, Pichia kluyveri only uses the glucose in the wort and have the ability of converting this substrate into a high concentration of specific flavor compounds. The fermentation product contained the flavor compound isoamyl acetate which is a key flavor in beer.

WO2020/035268A1 (Chr. Hansen A/S, Denmark) discloses using Pichia kluyveri to reduce earthy flavor caused by geosmin, a typical compound in root vegetable juices.

None of these patents describes fermentation of dairy or dairy analogue products or how to further modify the substrates utilized by Pichia kluyveri for improvements.

The present invention is suitable for preparing fermented food products where the fermentation is carried out in a controlled manner, i.e., with defined inoculum and processing conditions. This is in contrast to spontaneous fermentation where the fermentation is allowed to occur naturally by undefined ambient, wild microorganisms. Spontaneous fermentation gives inconsistent results, and often leads to the undesired drawbacks of spoilage, off-flavor or ethanol formation.

The invention is based on the surprising discovery that Pichia kluyveri is able to grow on lactic acid, but without formation of aroma, and that to allow aroma development, combination of lactic acid bacteria and specific carbohydrate(s) must be used together. It has further been discovered that co-fermentation with lactic acid bacteria in the presence of sucrose, fructose and glucose can allow flavor development and/or reduces ethanol formation, where sucrose and fructose give even better results.

Furthermore, propyl acetate formation can be detected in samples using the invention described herein, with highest amount observed when fructose was used. This has not been described before in connection with Pichia kluyveri.

As regards sugar fermentation, Pichia kluyveri is not able to utilize sucrose but fructose or glucose. In terms fructose or glucose, it was surprisingly discovered that although Pichia kluyveri can grow in the presence of fructose or glucose, no aroma was formed unless lactic acid bacteria were also present. Therefore, aroma formation requires the addition of lactic acid bacteria. Yeast growth as such does not necessarily indicate aroma formation. As mentioned, Pichia kluyveri is able to grow in the presence of fructose and glucose but does not synthesize flavor compounds. Therefore, for the first time, the inventor identified the synergistic effect of using Pichia kluyveri combined with lactic acid bacteria, and the carbohydrates sucrose, fructose and/or glucose as supplementing substrate for the co-culture leading to aroma formation by Pichia kluyveri. None of this has been described before or suggested by prior arts.

In a first aspect, the present invention provides uses of Pichia kluyveri, lactic acid bacteria and one or more carbohydrates selected from the group consisting of sucrose, fructose and glucose to increase aroma in a fermented food product, including dairy products and dairy analogue product. Preferably, the carbohydrate is sucrose or fructose.

Provided herein are also uses of Pichia kluyveri, lactic acid bacteria and one or more carbohydrates selected from the group consisting of sucrose, fructose and glucose to prepare fermented food product, including dairy products and dairy analogue product.

In some embodiments, the carbohydrate(s) are added before, at the start of, or during fermentation. The expression “at the start of the fermentation” means shortly before, at the same time as, or shortly after addition of Pichia kluyveri to the milk base. Here, the term “shortly” means less than 30 minutes. The expression “during the fermentation” means at any time during the fermentation after the start and before the end of the fermentation.

In some embodiments, a composition comprising at least one Pichia kluyveri strain, at least one lactic acid bacteria strain, and, optionally, sucrose, fructose and/or glucose, are added before fermentation.

Preferably, the lactic acid bacteria is homofermentative lactic acid bacteria. Homofermentative lactic acid bacteria are known in the art to produce lactic acid as primary metabolite. Homofermentative bacteria may be selected from Streptococcus thermophilus, Lactobacillus delbruckii subsp. bulgaricus, Lactobacillus acidophilus, Lactobacillus helveticus, and Pediococcus.

In one embodiment, the present invention provides a method for producing fermented food product, comprising:
providing a substrate which is a milk base which can be plant milk base and/or animal milk base
adding at least one Pichia kluyveri strain such as DSM 28484 and at least one lactic acid bacteria strain which is optionally a homofermentative lactic acid bacteria strain to the substrate, which is optionally prepared from legumes, nuts or cereal
fermenting the substrate, preferably until the pH is below 4.6, in the presence of one or more carbohydrate(s) selected from the group consisting of sucrose, fructose and glucose,
obtaining fermented food product.

Such compositions are described in more details in later sections of the present application.

Preferred Pichia kluyveri include Pichia kluyveri strain 1 (DSM 28484), Pichia kluyveri strain 2 (PK-KR1) and Pichia kluyveri strain 3 (PK-KR2), as well as mutants obtainable therefrom.

Pichia kluyveri PK-KR1 is known and was deposited under the Budapest Treaty on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/022711.

Pichia kluyveri PK-KR 2 is known and was deposited under the Budapest Treaty on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/022712.

To carry out the methods of the present invention, a suitable starting material is provided. This can be a milk base of animal origin, such as cow milk. In some embodiments, milk base prepared from plant material can be used. This is referred to herein as “plant milk” or “plant milk base.” Plant milks are colloidal suspensions or emulsions consisting of dissolved and disintegrated plant constituents. The general outline of preparation is very similar. They are mostly prepared by grinding the raw material to make into slurry, and then by straining to remove coarse particles. For large scale production, the plant material can be soaked and wet milled to extract the plant milk, or alternatively, the raw material is dry milled and soluble material is extracted in aqueous media. The insoluble material is separated by filtering or decanting, followed by addition of desired ingredients for acceptable product formulation. Homogenization and pasteurization/UHT treatment are often carried out to improve suspension and stability. A particle size distribution in range of 5-20 μm would imitate cow's milk in appearance and consistency.

Depending on the plant type, raw material is pretreated. Techniques like dehulling, soaking and blanching are sometimes preferred or required. For example, blanching can be done to inactivate trypsin inhibitors and lipoxygenase that would produce off-flavors in soy milk and peanut milk. As another example, roasting of the raw material can often enhances the aroma and flavor of the final product.

Methods of preparing plant milks, such as peanut milk, rice milk, oat milk, sesame milk, coconut milk, almond milk, hemp milk, hazelnut milk, tiger nut milk, lupin milk and quinoa milk etc. are known (e.g., Sethi et al “Plant-based milk alternatives an emerging segment of functional beverages: a review.” Journal of food science and technology 53.9 (2016): 3408-3423).

In preferred embodiments, the plant milk comprises at least 1% protein, such as at least 1.5%, at least 2%, at least 2.5%, at least 3%, at least 4%, at least 5%, at least 6% or at least 7% protein.

In preferred embodiments, the plant milk comprises at least 2% fat, such as at least 3%, at least 4%, at least 5%, at least 6% or at least 7% fat.

Proteins and/or can be already be inherent in the plant milk or supplemented during processing.

Legume

The present invention is especially useful for fermentation of legumes. Preferred legumes include soybeans, peas, beans, lupins, lentils. Preferably, the legume is soy.

The inventor observed that fermentation base prepared from soybeans contain almost no carbohydrates and is therefore a poor base for fermentation by Pichia kluyveri. Therefore, the base should be modified for the fermentation by Pichia kluyveri.

The inventor has also observed that only very trace amount of alcohol was produced by Pichia kluyveri when fermenting soymilk, making it ideal for dairy analogue where ethanol production should be minimal. In a preferred embodiment, the food product is a legume-based dairy alternative product, such as soy yogurt analogue.

In preferred embodiments, the fermented dairy or dairy analogue products have an alcohol content of less than 5000 ppm, such as less than 4000 ppm, such as less than 3000 ppm, such as less than 2000 ppm, such as less than 1000 ppm, such as less than 900 ppm, such as less than 800 ppm, such as less than 700 ppm, such as less than 600 ppm, such as less than 500 ppm.

Dairy analogues prepared from soymilk has been known for some time. However, it is always faced with the challenge of beany flavor, something not familiar to the Western consumers and is a barrier for consumption. The present inventor found that the flavor and taste of soy-based dairy analogues can be greatly improved by using Pichia kluyveri, where more aroma is created if appropriate substrates are supplemented. Aroma formation was also found in other plant milks (such as nut-based and cereal based plant milk) as well as in dairy products.

The present application provides methods of preparing fermented food product from soy material. For example, soymilk can be provided as starting material. Soymilk has been consumed widely and methods of preparation are known to a skilled person. They are for example described in Chapter 5 and 11 in Shurtleff, William, and Akiko Aoyagi. Tofu & Soymilk production: A Craft and Technical Manual. Vol. 2. Soyinfo Center, 2000.

Soymilk is generally made by soaking the soybeans, grinding soaked soybeans to obtain a slurry, straining the slurry to obtain soymilk. Soymilk can be extracted from the slurry before or after cooking.

Presently known methods for removing beany flavor include 1) vacuum treatment at high temperature which results in stripping off of most volatile compounds, 2) hot grinding method where soaked soybeans were ground with boiling water or steam to obtain a slurry at a temperature of 80 C, which is then kept at this temperature for 10 min in order to inactivate lipoxygenase, and 3) pre-blanching method where soaked soybeans were blanched in boiling water to inactivate lipoxygenase. Present invention represents an alternative strategy to improve the organoleptic properties of the fermented products originated from the plant material.

In other preferred embodiments, the legume source is peanut and the fermentation base is peanut milk. Peanut milk can be obtained by wet grinding and techniques including defatting, roasting, alkali soaking, steaming, as described by Lee et al. “Chemical, physical and sensory characteristics of peanut milk as affected by processing conditions.” Journal of Food Science 57.2 (1992): 401-405, by Diarra et al. “Peanut milk and peanut milk based products production: a review.” Critical reviews in food science and nutrition 45.5 (2005): 405-423, and by Galvez et al. “Optimization of Processing of Peanut Beverage.” Journal of Sensory Studies 5.1 (1990): 1-17.

Nuts

The present invention is especially useful for fermenting plant material prepared from nuts. Provided herein are methods of obtaining fermented dairy analogue product from nuts, comprising providing a substrate comprising plant material derived from nuts, fermenting the substrate with Pichia kluyveri in the presence of lactic acid and one or more carbohydrates selected from the group consisting of and sucrose, fructose and glucose.

In preferred embodiments, plant milk prepared from nuts are used as starting material. Preferred nuts include coconut, almond, cash, and walnut. Nut milk has a profile of healthy fatty acids and carbohydrates with low glycemic index as well as constituents of vitamins, antioxidants and dietary fiber. It may be made from almond, cashew nut, coconut and the like.

Preferably, the plant material is coconut milk or coconut cream prepared from coconut meat. Coconut milk is the aqueous extract of the solid coconut endosperm which may optionally include some coconut water. It is the white, opaque protein-oil-water emulsion obtained by pressing grated or comminuted solid coconut endosperm. Methods of preparation are known, for example as described in Cancel, L. E. “Coconut food products and bases.” Woodroof, J G Coconuts (1970). Extraction processes were reviewed by Seow et al. “Coconut milk: chemistry and technology.” International journal of food science & technology 32.3 (1997): 189-201. Coconut cream contains less water than coconut milk and the higher fat content gives it a smooth, thick and rich consistency.

Often, proteins are added to the fermentation base containing coconut milk and, in many cases, pea proteins or fava bean proteins are used. This may lead to the undesirable beany flavor which is a barrier to consumption.

The present inventor found that the flavor and taste of coconut-based dairy analogues can be improved by the use of Pichia kluyveri, where the product is perceived to be more aromatic than samples prepared with a kefir yeast (Example 4).

In a preferred embodiment, the fermented food product is a nut-based dairy alternative product such as coconut yogurt analogue.

In other embodiments, nut milk can be prepared from almond. Like soy milk, almond milk is a rich creamy milky white liquid which display similarities to cow milk in appearance and consistency. It is a nutrient dense product and an excellent source of vitamin E in the form of alpha-tocopherol and manganese. Almond milk can be obtained for example from extracted from dehulled almonds by soaking in water, wet-milling and straining the slurry. Methods for preparing almond milk is known to a skilled person for example as described in U.S. Pat. No. 5,656,321. Homogenization is often carried out, for example as described in Briviba et al. “Ultra high pressure homogenization of almond milk: Physico-chemical and physiological effects.” Food Chemistry 192 (2016): 82-89.

Cereal

The present invention is also useful for the fermentation of cereals. Provided herein are methods of obtaining fermented dairy analogue product from cereals, comprising providing a substrate comprising plant material derived from cereals, fermenting the substrate with Pichia kluyveri in the presence of lactic acid and one or more carbohydrates selected from the group consisting of sucrose, fructose and glucose.

Preferred cereals include oat, wheat, rye and millet. More preferably the cereal is oat.

The present inventor found that the flavor and taste of cereal-based dairy analogues can be improved by the use of Pichia kluyveri and lactic acid bacteria, where more aroma is created using suitable carbohydrates provided herein.

In a preferred embodiment, the food product is a cereal-based dairy alternative product, such as oat yogurt analogue.

Preferably, the plant material used as starting material is oat milk. Oat milk has a creamy texture similar to cow milk. Methods of preparing oat milk are known, for example as described by Deswal et al. “Optimization of enzymatic production process of oat milk using response surface methodology.” Food and bioprocess technology 7.2 (2014): 610-618 and U.S. Pat. No. 5,686,123. Since starch constitutes the major portion of the oats, application of heat, starch begins to gelatinize and liquid milk tends to attain a gel like consistency with high viscosity leading to its lower acceptability. Enzymatic hydrolysis (e.g., with alpha amylase) is often used to prevent gelatinization during thermal treatment and provide glucose to increase the sweetness perception.

Methods in accordance with the present invention comprise adding Pichia kluyveri and one or more lactic acid bacteria to the substrate. It should be understood that one or more Pichia kluyveri strains, such as 2, 3, 4, 5 or more strains can be added. As used herein, the term “strain” has its common meaning in the field of microbiology and refers to a genetic variant of a yeast or bacterium. As used herein, a Pichia kluyveri strain means a genetic variant of Pichia kluyveri, which can be determined by a skilled person based on the genetic makeup.

Likewise, it should be understood that one or more lactic acid bacteria strains, such as 2, 3, 4, 5 or more strains can be added.

The microorganisms are added to the substrate in an amount which is sufficient to initiate and maintain fermentation. The skilled person is able to determine suitable concentrations of inoculation using routine methods and in view of the current description and examples.

In preferred embodiments, Pichia kluyveri is inoculated in a concentration of at least 1×10⁴ CFU/ml, such as at least 5×10⁴ CFU/ml, such as at least 1×10⁵ CFU/ml, such as at least 5×10⁵ CFU/ml, such as at least 1×10⁶ CFU/ml, such as at least 5×10⁶ CFU/ml.

In preferred embodiments, the lactic acid bacteria are inoculated in a concentration of at least 1×10⁴ CFU/ml, such as at least 5×10⁴ CFU/ml, such as at least 1×10⁵ CFU/ml, such as at least 5×10⁵ CFU/ml, such as at least 1×10⁶ CFU/ml, such as at least 5×10⁶ CFU/ml.

Furthermore, the Pichia kluyveri and lactic acid bacteria added may be in frozen, liquid or dried form, including e.g. freeze-dried form and spray/fluid bed dried form, or frozen or freeze-dried concentrates.

In preferred embodiments, the Pichia kluyveri yeast cells can be hydrated or dehydrated. Examples of hydrated cells includes baker's yeast cake, compressed yeast and cream yeast. Examples of dehydrated cells include instant dry yeast, active dry yeast (ADY), and partially dried compressed yeast.

The term “compressed yeast” refers herein to a yeast with a dry matter content of between 35% and 90% (w/w) conventionally produced by propagation of yeast in a fermenter followed by concentration, filtration, extrusion and optionally partial drying on a drier, such as a fluid bed drier. In some embodiments, the dry matter content is between 30% and 45%, such as between 30% and 40% or between 35% and 45%.

The term “cream yeast” herein refers to liquid yeast with a dry matter content of below 28% (w/w) conventionally produced by propagation of yeast in a fermenter followed by concentration by centrifugation.

The term “active dried yeast” or “ADY” refers herein to yeast with a dry matter content of more than 90% (w/w) conventionally produced by propagation of yeast in a fermenter followed by concentration, filtration, extrusion and drying on a fluid bed drier.

Thus, the term “partially dried compressed yeast” refers herein to a yeast with a dry matter content of between 45% to 90% (w/w) produced by propagation of yeast in a fermenter followed by concentration, filtration, extrusion and partial drying on a drier, such as a fluid bed drier.

The method in accordance with the present invention comprises the step of fermenting the substrate that contains Pichia kluyveri and lactic acid bacteria. Fermentation of the substrate is preferably carried out by controlled fermentation in sterile settings. During the fermentation process, a skilled person in the art is able to adjust other fermentation parameters known to him in order to achieve the desired end-product.

Fermentation of the substrate by Pichia kluyveri is characterized by the presence of lactic acid and one or more carbohydrates selected from the group consisting of sucrose, fructose and glucose. Embodiments include, but are not limited to, 1) one or more lactic acid bacteria strains and sucrose, 2) one or more lactic acid bacteria strains and fructose and 3) one or more lactic acid bacteria strains and glucose and 4) one or more lactic acid bacteria strains and a mixture of at least two or all of the following: sucrose, fructose and glucose.

Fructose and glucose can be provided directly, or indirectly by providing suitable sugars for the lactic acid bacteria strain to metabolize, thereby providing fructose and glucose. This can be selected depending on the fermentation profile of the lactic acid bacteria.

The inventors show that Pichia kluyveri does not require high amount of sugars in the fermentation medium for aroma formation, as shown in Example 3.

Preferably, the fermentation base comprises about 1-10% (wt) sucrose, fructose and glucose. In this, it has been observed that high amount glucose may cause stress for yeast growth. Therefore, it is preferred that the glucose concentration is below 20%, such as below 15% or below 10% (wt).

Lactic acid bacteria and the carbohydrate(s) can be added before, at the start, or during the fermentation. In some embodiments, they are added before or at the start of the fermentation, preferably together as a starter culture composition.

Afterwards, the fermentation base is subjected to a suitable condition and the fermentation process begins and continues for a period of time. A person of ordinary skill in the art knows how to select suitable process conditions, such as temperature, oxygen, and the process time.

The fermentation can be carried out at a temperature of between 20-38° C. In preferred embodiments the fermentation is carried out at a temperature of between 22 and 35° C., such as between 25-32° C., such as 28-30° C. In some preferred embodiments the fermentation temperature is between 23-26° C.

The fermentation condition using Pichia kluyveri may be semi-anaerobic. The fermentation starts aerobically and proceeds anaerobically after all oxygen is consumed. The fermentation condition using Pichia kluyveri may also be aerobic. A skilled person may choose the conditions suitable for the intended type of products.

A pH which is higher than 4.6 can be considered unsafe for fermented food products. Using the methods of the present invention, it is possible to reach a pH which is below 5, such as pH 4.6 or lower, within a short period of time. Preferably, the fermentation with Pichia kluyveri and lactic acid bacteria is carried out for 12 hours, such as for 18 hours, such as for 24 hours, such as for 36 hours, such as for 48 hours.

In one embodiment, both lactic acid and fructose are present in the fermentation base. Fructose can be provided by directly or indirectly, for example by adding at least one lactic acid bacteria strain and sucrose to the substrate and fermenting the substrate, wherein the lactic acid bacteria strain is able to utilize the glucose that forms part of the sucrose, and release fructose.

A skilled person is able to select suitable lactic acid bacteria based on its sugar fermentation pattern, which can be determined with methods known in the art. Differences in sugar requirements among LAB strains has been used for enumeration, selection, and identification (Kandler, O., and N. Weiss. “Regular nonsporing Gram positive rods. Section 14 In: Garrity, G. (Ed) Bergey's Manual of Systematic Bacteriology.” Springer, New York 2 (1986): 1208-1260 and reviewed by Kandler “Carbohydrate metabolism in lactic acid bacteria.” Antonie van Leeuwenhoek 49.3 (1983): 209-224). Generally, sugar fermentation is detected by acid formation from a sugar given in growth media.

In other embodiments, the plant material is treated to provide fructose and/or glucose, for example by enzymatic hydrolysis.

In one embodiment, both lactic acid and glucose are present in the fermentation base. Glucose can be provided by directly or indirectly, for example by adding at least one lactic acid bacteria strain and sucrose to the substrate and fermenting the substrate, wherein the lactic acid bacteria strain is able to utilize the fructose that forms part of the sucrose, and release glucose.

Fermentation can be terminated by any suitable methods known in the art, including cooling down, preferably to below 4° C. Optionally, pasteurization of the final product may be carried out to prolong shelf life.

It has further been discovered that the combination of lactic acid bacteria and fructose or glucose can lead to the highest formation of propyl acetate, in particular in soymilk. Propyl acetate is a compound known by its pleasant, bittersweet flavor reminiscent of pear on dilution (Burdock, G. A. (ed.). Fenaroli's Handbook of Flavor Ingredients. 6th ed. Boca Raton, Fla. 2010, p. 1739).

In a further aspect the present invention provides yeast-fermented dairy products or dairy analogue products. Products prepared according to the present invention have improved flavor profiles and comprises Pichia kluyveri and lactic acid bacteria. Pichia kluyveri strain 1 (DSM 28484), Pichia kluyveri strain 2 (PK-KR1), Pichia kluyveri strain 3 (PK-KR2), or mutants thereof or any of the combinations thereof can be used. The two Pichia kluyveri strains PK-KR1 and PK-KR2 were originally disclosed in WO2009110807, where it was described that they can be used to increased thiol levels (3 MH and 3 MHA) in wine fermentation process. PK-KR1 and PK-KR2 were also used with different hop varieties to brew beer (WO2013030398) as well as low-alcohol or alcohol-free beer (WO2014135673).

Furthermore, with the methods disclosed herein it is possible to obtain dairy analogue products with high viscosity. Higher viscosity can be observed if the milk base used is rich in fat. For example, the yogurt analogues prepared from soy and oat have a viscosity of at least around 30-40 Pa. For coconut yogurt analogues viscosity of more than 100 Pa or even 200 Pa can be obtained.

Starter Culture Composition

The present invention also provides a starter culture composition comprising at least one Pichia kluyveri strain and at least one lactic acid bacteria strain, such as at least 2, at least 3, at least 4 or at least 5 strains. The term “starter culture” refers to a composition comprising live microorganisms that are capable of initiating or effecting fermentation of organic material after being cultivated in a separate starter medium for obtaining a high-density culture.

The inventor discovered that the synergism between these microorganisms in the presence of certain carbohydrates contributes positively to the flavor profile of fermented food products compared to using Pichia kluyveri alone.

The yeast and bacteria may be supplied either as frozen or freeze-dried cultures for bulk starter propagation or as so-called “Direct Vat Set” (DVS) cultures, intended for direct inoculation into a fermentation vessel or vat for the production of a fermented product, such as a fermented dairy or dairy analogue products. The starter culture composition is preferably in a frozen, dried or freeze-dried form, e.g. as a Direct Vat Set (DVS) culture. However, the composition may also be a liquid that is obtained after suspension of the frozen, dried or freeze-dried cell concentrates in a liquid medium such as water or PBS buffer. Where the composition of the invention is a suspension, the concentration of viable cells is in the range of 10⁴ to 10¹² cfu (colony forming units) per ml of the composition including at least 10⁴ cfu per ml of the composition, such as at least 10⁵ cfu/ml, e.g. at least 10⁶ cfu/ml, such as at least 10⁷ cfu/ml, e.g. at least 10⁸ cfu/ml, such as at least 10⁹ cfu/ml, e.g. at least 10¹⁰ cfu/ml, such as at least 10¹¹ cfu/ml.

Preparation of the different forms of start culture comprising yeast are known in the art and for example described in WO2011/134952 (Chr. Hansen A/S). Pichia kluyveri can be grown in in a fermenter and concentrated. Additionally, cryoprotectants can be added to maintain the viability of the yeast under a low temperature. Buffering agents and growth stimulating nutrients, preservatives or other carriers known in the art may be included.

In a preferred embodiment the starter culture contains at least 10⁴ CFU/g colony forming units (CFU)/g of Pichia kluyveri, such as at least 10⁵ CFU/g, such as at least 10⁶ CFU/g, such as at least 10⁷ CFU/g, such as at least 10⁸ CFU/g, such as at least 10⁹ CFU/g, such as at least 10¹⁰ CFU/g, such as at least 10¹¹ CFU/g, such as at least 10¹² CFU/g, such as at least 10¹³ CFU/g.

In a preferred embodiment the starter culture contains at least 10⁴ colony forming units (CFU)/g of lactic acid bacteria, preferably Streptococcus thermophilus or Lactobacillus spp., such as at least 10⁵ CFU/g, such as at least 10⁶ CFU/g, such as at least 10⁷ CFU/g, such as at least 10⁸ CFU/g, such as at least 10⁹ CFU/g, such as at least 10¹⁰ CFU/g, such as at least 10¹¹ CFU/g, such as at least 10¹² CFU/g, such as at least 10¹³ CFU/g of lactic acid bacteria.

In preferred embodiments, the present application provides starter culture compositions comprising Pichia kluyveri and lactic acid bacteria strain(s) as well as carbohydrate(s) selected from the group consisting of sucrose, fructose and glucose.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Deposit and Expert Solution

The applicant requests that a sample of the deposited microorganism stated below may only be made available to an expert, until the date on which the patent is granted.

The applicant deposited the Pichia kluyveri strain 1 on 5 Mar. 2014 at Leibniz Institute DSMZ—Deutsche Sammlung von Mikroorganismen and Zellkulturen GmbH (DSMZ), Inhoffenstr. 7B, D-38124 Braunschweig, and received the accession No.: DSM 28484.

The deposit has been made under the conditions of the Budapest Treaty on the International Recognition of the Deposit of Microorganisms for the Purposes of Patent Procedure.

### EXAMPLES

### Example 1

Sample Preparation

Organic unsweetened soy milk (NATURLI′ Foods A/S, Denmark) was used as starting material. Different amount of glucose (Cargill), sucrose (Nordic sugar) and fructose (Hamburg Fructose GmbH International) were added either in the presence of added lactic acid or lactic acid bacteria (FDVS-YF-L01, containing Streptococcus thermophilus; Chr. Hansen Denmark) as shown in Table 1. The amount of lactic acid selected was 0.5%, which is the same amount expected to be produced by LAB in vegetable bases such as soy milk. The amount of added glucose, fructose and sucrose was determined such that all samples had the same sweetness perception equal to 5 based on the sweetness index, with sucrose equal to 1, fructose equal to 1.65 and glucose to 0.69. Soymilk was pasteurized (90° C. for 20 minutes) after adding the sugars and/or the lactic acid to avoid contamination. The base was then cooled to fermentation temperature, 30° C. Pichia kluyveri strain 1 was inoculated in the amount of 1×10⁶ cfu/g and the lactic acid bacteria was inoculated as in the amount shown in Table 1 at the start of fermentation. In this example, fermentation was performed in 5 L scale. After 37.8 h, the coagulum was broken with a perforated disk. Then, a cooling step to 25° C. followed by a mechanical post-treatment (2 bars back-pressure; FH Scandinox A/S, Tarm, Denmark) was performed to allow the smoothing process. The samples were collected in 120 ml plastic cups and stored at 6° C. for further analysis.

TABLE 1
Experimental design of Example 1

| | Carbohydrate | Lactic acid | Streptococcus | pH adjusted |
|---|---|---|---|---|
| Sample | (wt %) | (wt %) | thermophilus (wt %) | to 4.55 |
| Soy 1 | 0% | 0.5% | 0% | No |
| Soy 2 | 7.2% Glucose | 0% | 0% | No |
| Soy 3 | 3% Fructose | 0% | 0% | No |
| Soy 4 | 5% Sucrose | 0% | 0.02% | No |
| Soy 5 | 3% Fructose | 0% | 0.02% | No |
| Soy 6 | 5% Sucrose | 0.5% | 0.02% | No |
| Soy 7 | 7.2% Glucose | 0% | 0% | Yes |
| Soy 8 | 3% Fructose | 0% | 0% | Yes |

Fermentation Profile

Fermentation profile was recorded with an Axone system (InLab® Sensors, Mettler Toledo).

Pichia kluyveri Enumeration

Enumeration of Pichia kluyveri was performed on YGC agar petri dishes by a spread method. Briefly, YGC agar was melted at 99° C. in a water bath for 45-55 minutes, then cooled to approximately 52° C. and placed in a petri dishes for approximately 1 hour before use. A dilution series in peptone water was performed, followed by a spreading of 100 μl of the 10⁻⁴ dilution. A control plate was also used. Afterwards, the sample was absorbed by the agar, and the plate was inverted and incubated in a plastic bag at 20° C. for 3 days. After incubation the colonies were counted and the results were reported in CFU/g. CFU/g was calculated as average of duplicates plate and divided by the grams weight out multiply with the dilution factor (Equation 1).

$$\frac{CFU}{G} = ( \frac{Plate 1 + PLate 2}{2} ) * ( \frac{1}{g * dilution factor} ) \qquad ( Equation 1 )$$

Volatile Organic Compounds (VOCs) Analysis

This method exploits the volatility by performing analysis on the volatile fraction by Headspace (HS) (Perkin Elmer TurboMatrix110 Headspace sampler, Perkin Elmer, Denmark) Gas chromatography (GC) and Flame Ionisation Detector (FID) (Perkin Elmer Autosystem XL GC coupled, Perkin Elmer, Denmark). HS-GC-FID was used to quantify volatile compounds (acetaldehyde, acetone, 3-methyl-butanal, ethanol, diacetyl, butan-1-01 and acetoin). Samples were prepared by adding 200 μl of 4N H₂SO₄ to 1 ml fermented sample and immediately analyzed by HSGC. The GC was equipped with a HP-FFAP column (25 m×0.20 mm×0.33 mm, Agilent Technologies, Germany). The injector was hold at 180° C. Samples were heated for 36.5 minutes at 70° C. in the headspace autosampler before injection (needle temperature: 180° C.). Helium was used as the carrier gas with a pressure of 32 psi. Transfer line was hold at 210° C. The SOF-program works as follows: after starting at 60° C., the oven temperature was raised after 2 minutes from 60° C. to 230° C. at 45° C./min and was finally held at 230° C. for 0.5 min. The FID temperature was kept constant at 220° C. with hydrogen and air flows of 45 ad 450 mL/min respectively. The FID signal was attenuated at −6 with an offset 5 mV. Data were processed by Chromeleon software (version 7.2.7, Thermo Fisher Scientific Inc., Denmark).

For screening, Dynamic Headspace coupled to Gas chromatography and Mass Spectrometry detector (DHS-GC-MS) was used to identify and semi-quantify (as relative intensities) the volatile composition present in the samples. In a 20 ml headspace vial (ML-33015SPME, Mikrolab Aarhus A/S, Arhus, Denmark) 0.2 mL 200 μL 2M H₂SO₄ and 1 g of sample was added. Vial capped (ML-33041C, Mikrolab Aarhus A/S, Arhus, Denmark). Stored at max. −18° C. until analysis. Samples were analyzed using a gas chromatograph (Agilent 7890B, Agilent Technologies, Denmark) coupled to a single quadropole mass spectrometer (Agilent 5977A, Agilent Technologies, Denmark) after 30 min. extraction at 30° C. of volatiles from the headspace above the sample in the vial using dynamic headspace extraction onto a TenaxTA tube (Gerstel #020810, MSCI, Skovlunde, Denmark). The TenaxTA tube was desorbed in a termal desorption unit (TDU, Gerstel, MSCI, Skovlunde, Denmark) at 270° C. for 5 min. The volatiles were arrested in the cooled inlet at 10° C. in a TenaxTA liner (Gerstel #012438, MSCI, Skovlunde, Denmark). Volatiles desorbed to the GC column by rapid heating of the TenaxTA liner to 270° C. in splitless mode: Pressure: 170 kPa, Total Flow: 40 ml/min Transfer mode: Splitless time: 2 min. Volatiles were separated on an apolar column (DB-5MS UI 30 m×0.25 mm×1 μm, Agilent #122-5533UI, Agilent Technologies, Denmark) using constant pressure at 170 kPa resulting in a start flow of 2.6 ml/min@32° C. ending at 0.85 ml/min@325° C. Oven temperature program was as follows: 32° C. for 2 min, raised at 10° C./min to 102° C., 5° C./min to 145° C., 15° C./min to 200° C., 20° C./min to 325° C., total run time of 27.5 min. The mass spectrometer operated in electronic impact mode at −70 eV and the analyzer was scanning from 29-209 amu. Height response was used for calculation of the semi quantitative results employing MassHunter software (Version 10.0, Build 10.0.707.0, Agilent Technologies, Denmark).

Results

Fermentation Profile

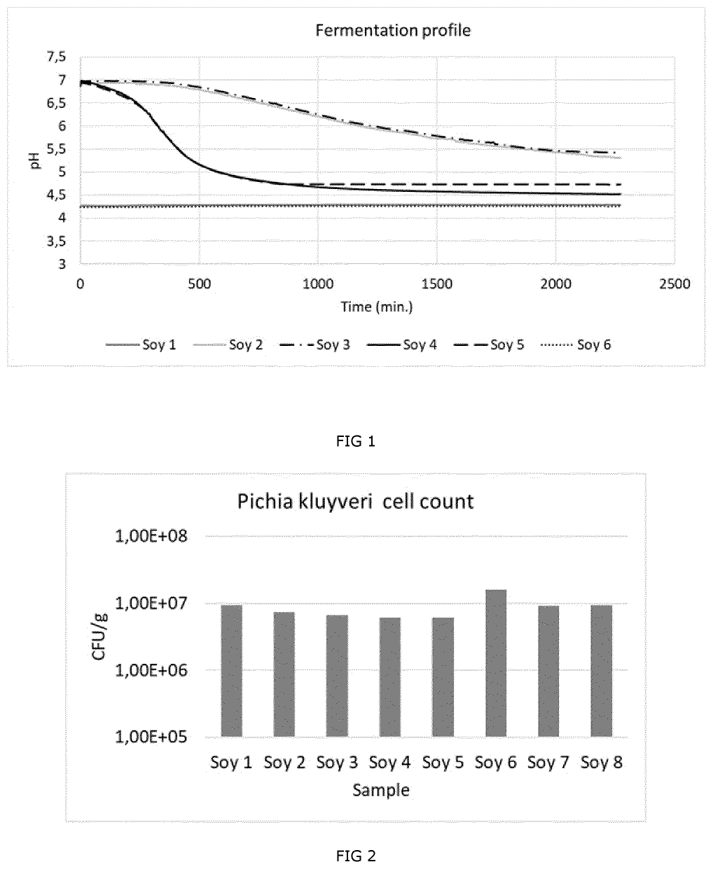

The fermentation profiles of the above mentioned soy samples (Table 1) are reported in FIG. 1. As shown, rapid fermentation can be achieved by using lactic acid bacteria in the presence of sucrose or fructose (Soy 4 and Soy 5), where the pH dropped to approximately 4.5 in about 17 hours. In contrast, as shown in samples Soy 2 and Soy 3, if only glucose and fructose were added but not lactic acid bacteria, Pichia kluyveri ferments slowly, reaching a pH of 5.5 after 37 hours.

This demonstrates that the combined use of Pichia kluyveri, lactic acid bacteria in the presence of sucrose or fructose leads to rapid fermentation, resulting in a pH range which is accepted for fermented food products for food safety.

Growth of Pichia kluyveri

Pichia kluyveri growth was evaluated in the samples after 1 day of storage and shown in FIG. 2. Two additional samples, Soy 7 and Soy 8 were prepared, respectively from Soy 2 and Soy 3, by adjusting the pH with lactic acid to 4.55 in order to do not influence the sensory evaluation, as acidity can influence the descriptors.

As reported in FIG. 2, Pichia kluyveri was able to grow in all soy samples. It was observed that Pichia kluyveri grows the fastest in samples containing lactic acid (Soy 1 and Soy 6). This can also be observed in the samples Soy 7 and Soy 8, where the addition of lactic acid led to a higher cell count compared to the original samples where the pH was not adjusted (Soy 2 and Soy 3).

This demonstrates that lactic acid can be supplied to Pichia kluyveri to further enhance its growth. This may be useful in products where the cell count of the microorganisms is important, such as kefir products.

Sensory Evaluation

A sensory evaluation was performed on all the samples after 2 days of storage. Two main attributes, sweet and fruity perception, were evaluated. The assessors were asked to rank the samples from the lowest (6) to the highest (1) sweetness and fruity intensity, 6 indicating the least sweet/fruity and 1 indicating the most sweet/fruity.

TABLE 2
Ranking of sweetness and fruitiness perception in the fermented soy
samples

| | | Lactic | Streptococcus | | |
|---|---|---|---|---|---|
| | Carbohydrate | acid | thermophilus | | |
| Sample | (wt %) | (wt %) | (wt %) | Sweetness | Fruitiness |
| Soy 1 | 0% | 0.5% | 0% | 6/absent | 6/absent |
| Soy 2 | 7.2% Glucose | 0% | 0% | 4 | 4-5 |
| Soy 3 | 3% Fructose | 0% | 0% | 5 | 4-5 |
| Soy 4 | 5% Sucrose | 0% | 0.02% | 1 | 2 |
| Soy 5 | 3% Fructose | 0% | 0.02% | 3 | 3 |
| Soy 6 | 5% Sucrose | 0.5% | 0.02% | 2 | 1 |

Regarding sweetness, the best performing samples were fermented with Pichia kluyveri, lactic acid bacteria in the presence of sucrose and fructose (Soy 4, Soy 6 and Soy 5). In detail, Soy 4 (5% sucrose and lactic acid bacteria) was perceived to be the sweetest, followed by Soy 6 (5% sucrose, lactic acid bacteria and 0.5% of lactic acid), and then Soy 5 (3% fructose and lactic acid bacteria).

Samples without lactic acid bacteria (Soy 2 and Soy 3) were perceived to be the least sweet despite having the same sweetest index.

In Soy 1, where only 0.5% of lactic acid was added, therefore without sugars and lactic acid bacteria, sweetness was not detected despite growth of Pichia kluyveri. This clearly indicates that lactic acid bacteria are needed for the sweetness perception.

Regarding fruitiness, Soy 6 (5% sucrose, lactic acid bacteria and 0.5% lactic acid) had the highest fruity perception, followed by Soy 4 (5% sucrose and lactic acid bacteria) and then Soy 5 (3% fructose and lactic acid bacteria). In Soy 1, fruitiness was not perceived either, consistent with the observation for sweetness.

The outcome of the sensory evaluation demonstrates that best flavors can be achieved using Pichia kluyveri, lactic acid bacteria, in the presence of sucrose or fructose. Lactic acid may be added to enhance fruitiness. There is a synergistic effect by using Pichia kluyveri, lactic acid bacteria in the presence of certain sugars in aroma formation.

Furthermore, the presented data show that the high cell count is not necessarily linked to aroma formation (see Soy 1). The addition of lactic acid did not result in aroma development.

Volatile Organic Compounds (VOCs) Analysis

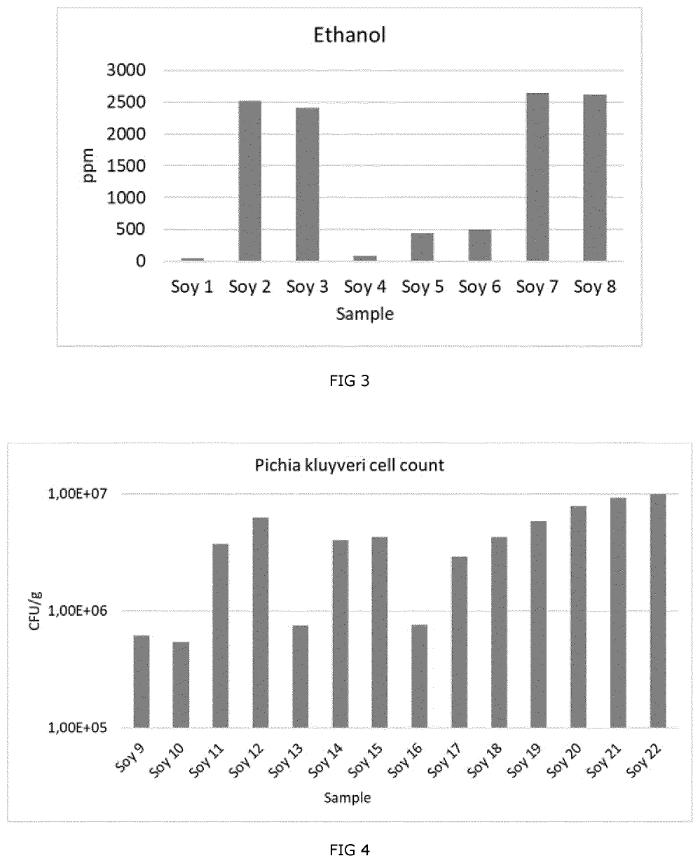

FIG. 3 shows the level of ethanol in all samples. It has been surprisingly found that with the addition of lactic acid bacteria, it is possible to reduce the ethanol formation by the yeast. As shown, lowest amount of ethanol was detected in Soy 4 (5% sucrose and lactic acid bacteria), Soy 5 (3% fructose and lactic acid bacteria) and Soy 6 (5% sucrose, and lactic acid bacteria and 0.5% lactic acid).

### Example 2

Sample Preparation

Organic unsweetened soy milk (NATURLI′ Foods A/S, Denmark) was used as starting material. Different amount of glucose (Cargill), sucrose (Nordic sugar) and fructose (Hamburg Fructose GmbH International) were added either in the presence of added lactic acid (Lactol Vinoferm, Brouwland, Belgium) or in the presence of lactic acid bacteria (FDVS-YF-L01, containing Streptococcus thermophilus; Chr. Hansen Denmark) as shown in Table 3. Soymilk was pasteurized (90° C. for 20 minutes) after adding sugars and/or lactic acid to avoid contamination. Pichia kluyveri strain 1 was inoculated in the amount of 1×10⁶ cfu/g and the lactic acid bacteria was inoculated as in the amount shown in Table 3 at the start of fermentation. In this example, fermentation was performed at 30° C. for 43.5 hours in 200 mL scale. After that, the coagulum was broken with a perforated disk and the samples were stored at 6° C. for further analysis.

TABLE 3
Experimental design of Example 2, and informal evaluation of
perceived aroma by smelling.

| | | Lactic | Streptococcus | |
|---|---|---|---|---|
| | Carbohydrate | Acid | thermophilus | |
| Sample | (Wt %) | (Wt %) | (Wt %) | Aroma |
| Soy 9 | 0% | 0% | 0% | Absent-Very Low |
| Soy 10 | 0% | 0.3% | 0% | Absent-Very Low |
| Soy 11 | 0% | 0.5% | 0% | Absent-Very Low |
| Soy 12 | 0% | 1% | 0% | Low |
| Soy 13 | 5% Sucrose | 0% | 0% | Absent-Very Low |
| Soy 14 | 5% Glucose | 0% | 0% | Low-Medium |
| Soy 15 | 5% Fructose | 0% | 0% | Low-Medium |
| Soy 16 | 25% Glucose | 0% | 0.02% | Absent-Very Low |
| Soy 17 | 5% Sucrose | 0% | 0.02% | Low |
| Soy 18 | 5% Glucose | 0% | 0.02% | High |
| Soy 19 | 5% Fructose | 0% | 0.02% | High |
| Soy 20 | 5% Sucrose | 0.5% | 0.02% | Low |
| Soy 21 | 5% Glucose | 0.5% | 0.02% | Medium-High |
| Soy 22 | 5% Fructose | 0.5% | 0.02% | High |

Pichia kluyveri Enumeration

One day after storage, Pichia kluyveri was enumerated according the method described in Example 1.

Informal Sensory Evaluation

Informal sensory evaluation to detect aroma formation was made by smelling them and grading the perceived aroma from absent to very low, low, medium, and high. Results are shown in Table 3.

Results

Growth of Pichia kluyveri

Pichia kluyveri growth was evaluated in all the samples after 1 day of storage and the results are shown in FIG. 4. It can be seen that lactic acid was able to enhance the growth of Pichia kluyveri, with the cell count positively correlate to the lactic acid added (see Soy 10, Soy 11 and Soy 12, containing 0.3%, 0.5% and 1% lactic acid, respectively).

In the presence of only the carbohydrate, higher cell count was seen when glucose or fructose was added (Soy 14 and Soy 15, containing 5% glucose and fructose, respectively). However, no growth was seen when 5% of sucrose was added (Soy 13). This indicates that Pichia kluyveri cannot use sucrose as a substrate. Furthermore, higher amount of glucose showed inhibition of Pichia kluyveri growth (Soy 16).

The limitation factor of not being able to utilize sucrose in the base was overcome by adding lactic acid bacteria (comparing Soy 13 to Soy 17). In this case, Pichia kluyveri showed growth in the presence of sucrose, reaching to a similar level as in the presence of glucose (comparing Soy 17 and Soy 14). In soy samples where neither carbohydrates nor lactic acid was added (Soy 9), Pichia kluyveri did not grow and the cell count dropped to approximately 6×10⁴ cfu/g.

Informal Sensory Evaluation

While Pichia kluyveri was able to grow well with the addition of lactic acid or suitable carbohydrates, little or no aroma formation was perceived. Surprisingly, for when lactic acid bacteria were added, in increase in aroma formation can be detected (comparing Soy 13-Soy 17, Soy 14-Soy 18, Soy 15-Soy 19).

### Example 3

Sample Preparation

Organic unsweetened soy milk (NATURLI′ Foods A/S, Denmark) was used as starting material. 5% of glucose (Cargill), sucrose (Nordic sugar), fructose (Hamburg Frutose GmbH International) or lactose (Infantose Sachsenmilch Lepperdorf GmbH) was added as shown in Table 4. After adding the sugars, soymilk was pasteurized (90° C. for 20 minutes) to avoid contamination. The bases were cooled to fermentation temperature, 30° C., and inoculated with 0.02% of lactic acid bacteria (FDVS-YF-L01, containing Streptococcus thermophilus; Chr. Hansen Denmark). Pichia kluyveri strain 1 was inoculated in the amount of 1×10⁶ cfu/g at the start of fermentation. In this example, fermentation was performed in 5 L scale. When the pH reached 4.55, the coagulum was broken with a perforated disk, a cooling step to 12° C. followed by a mechanical post-treatment (2 bars back-pressure; FH Scandinox A/S, Tarm, Denmark) was performed to allow the smoothing process. The samples were collected in 120 ml plastic cups and stored at 6° C. for further analysis.

TABLE 4
Experimental design of Example 3

| | | Streptococcus |
|---|---|---|
| | Carbohydrate | thermophilus |
| Sample | (wt %) | (wt %) |
| Soy 23 | 5% sucrose | 0.02% |
| Soy 24 | 5% fructose | 0.02% |
| Soy 25 | 5% glucose | 0.02% |
| Soy 26 | 5% lactose | 0.02% |

Sensory Evaluation: Descriptive Analysis

Nine judges participated in the test to evaluate the four samples obtained 7 days after storage. One training sessions was held to familiarize the panelists with the samples and to define the attribute list. For the evaluation, the judges were presented to the samples in randomized order following a Latin square design in two replicates. Attribute intensities were rated on a structured line scale with five compartments that was labeled with “none” on the left end and “a lot” on the right.

Statistical evaluation of the results for the intensity evaluation included three-way MANOVA (multivariate analysis of variance) with Wilks test to check overall sample differences and ANOVA (Analysis of variance) to find for which attribute there were significant differences, both considering the factors product, judge and replicate as well as their two-way interactions. The Least Significant Difference (LSD) test was used to detect significant differences among the product samples when the attributes had a significant product effect. A significance level of α=0.05 was selected for the study. ANOVAs were calculated for each attribute. The differences between the samples was calculated by means of a least significant difference test (LSD) test.

Multivariate data analysis and Pearson correlation between the volatile components and the sensory attributes in all tested samples, was performed with Simca 15 (Umetric, Sweden).

Volatile Organic Compounds (VOCs) Analysis

Seven days after storage, VOCs were analyzed according the method described in Example 1. Signal to noise ratio (SNR) is given.

Sugar Quantification

Seven days after storage, sucrose, glucose and fructose were quantified in all the samples by the AOAC 982.14, mod./HPAEC-PAD method (Eurofins).

Results

Sensory Evaluation: Descriptive Analysis

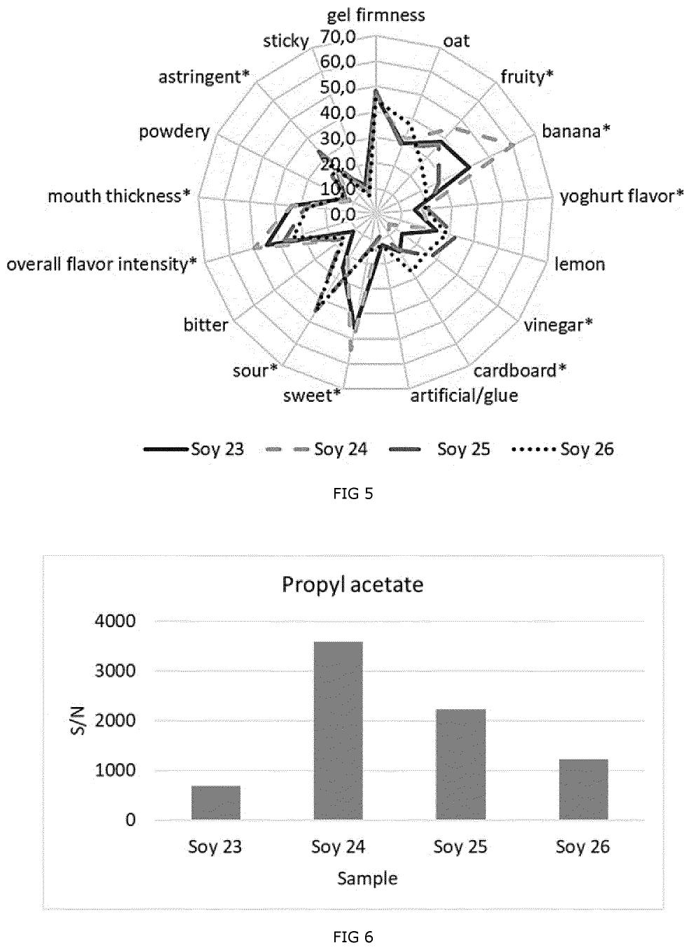

The outcome from the sensory evaluation is reported in Table 5, showing the mean values and grouping of the samples based on the least significant difference test (LSD) for the attributes that were found to be significantly different among the products, different letters indicate significant differences at p<0.05. The same data is depicted in a spider plot in FIG. 5.

As can be seen in FIG. 5, the sample containing fructose (Soy 24) have the highest fruity, banana, sweet and overall flavor intensity attributes, which is followed by sample containing sucrose (Soy 23). Moreover, those samples are also the ones being the lowest off-flavor, such as vinegar, cardboard and astringent attributes.

TABLE 5
Evaluation of different attributes

| | Soy 23 | Soy 24 | Soy 25 | Soy 26 |
|---|---|---|---|---|
| Attributes | (sucrose) | (fructose) | (glucose) | (lactose) |
| Gel firmness | 46.6 | 44.3 | 48.3 | 44.9 |
| Oat | 29.6 | 31.5 | 28.2 | 37.5 |
| Fruity | 38.3^{a} | 45.5^{a} | 36.5^{a} | 26.4^{b} |
| Banana | 41^{b} | 60.4^{a} | 27.6^{c} | 21.9^{c} |
| Yogurt flavor | 15.4^{b} | 18.5^{ab} | 20.3^{a} | 19.6^{ab} |
| Lemon | 24.7 | 21.7 | 32.7 | 29.1 |
| Vinegar | 12.9^{b} | 6.7^{c} | 27.6^{a} | 25^{a} |
| Cardboard | 17.4^{b} | 9.2^{c} | 16.9^{b} | 26.8^{a} |
| Artificial/Glue | 12.4 | 8.6 | 8.7 | 12.8 |
| Sweet | 45.9^{b} | 54.4^{a} | 14.6^{c} | 14.9^{c} |
| Sour | 24.8^{b} | 21^{b} | 44.8^{a} | 44.5^{a} |
| Bitter | 11.3 | 17 | 16.8 | 15.8 |
| Overall flavor intensity | 44.5^{ab} | 49.8^{a} | 37.6^{bc} | 33.8^{c} |
| Mouth thickness | 33.4^{a} | 33.4^{a} | 28.1^{b} | 27^{b} |
| Powdery | 13.7 | 11.4 | 13.7 | 14.6 |
| Astringent | 22.3b | 21.5b | 33.2a | 27.1ab |
| Sticky | 12 | 7.8 | 9.3 | 7.8 |

VOCs Analysis and Correlation with Sensory Evaluation

FIG. 6 shows the amount of propyl acetate (S/N)) in all four samples. The inventor has surprisingly observed that propyl acetate is present in high amounts in the samples fermented with lactic acid bacteria and fructose (Soy 24) and glucose (Soy 25). The Person correlation values obtained between propyl acetate and the sensory attributes showed a positive correlation between fruity and banana flavor (Table 6). This is in line with the finding of significantly higher scores for the banana flavor attribute in the sample containing fructose.

TABLE 6
Pearson correlations values between sensory
attributes and propyl acetate compound.

| Sensory Attributes | Propyl acetate |
|---|---|
| Fruity | 0.65 |
| Banana | 0.65 |
| Yoghurt flavor | 0.18 |
| Vinegar | −0.43 |
| Cardboard | −0.63 |
| Sweet | 0.39 |
| Sour | −0.36 |
| Overall flavor intensity | 0.56 |
| Mouth thickness | 0.28 |
| Astringent | −0.2 |

Sugar Quantification

Table 7 shows the amount of glucose, fructose, galactose and sucrose (g/100 g) measured in the samples Soy 23, Soy 24 and Soy 25 after fermentation. These samples contained 5% sucrose, fructose and glucose, respectively, at the start of the fermentation, which corresponds to 5 g per 100 g (Table 4).

For Soy 23, approximately 0.32 g/100 g of sucrose was consumed, and for Soy 24 about 0.33 g/100 g of glucose was consumed. This demonstrates the microorganisms do not require high amounts of sugar. Similar observation is made for Soy 25, where approximately 0.93 g/100 g glucose was consumed.

TABLE 7

| Sample | glucose | fructose | galactose | sucrose |
|---|---|---|---|---|
| Soy 23 (sucrose) | | | 0.04 | 4.64 |
| Soy 24 (fructose) | | 4.67 | | |
| Soy 25 (glucose) | 3.93 | 0.07 | | 0.07 |

### Example 4

Sample Preparation

The following three plant milk bases were provided to provide yogurt analogues:
Soy base (organic unsweetened soy milk (NATURLI′ Foods A/S, Denmark) with addition of 5% sucrose (Nordic sugar))
Coconut base (consisting of 57% of coconut milk (Aroy-D), 31.5% of natural coconut water (myCoco), 6.5% water, 4% starch (Clearam CH2020, Roquette) and 1% sucrose (Nordic sugar))
Oat base (obtained from 30% (wt) oat bran aqueous extraction and submitted to enzymatic treatment of partial hydrolysis and saccharification of starch, followed by heat treatment (Oatvita, Frulact, Portugal); the base contains 2.2% fat and approximately 4% protein and 10% glucose).

Bases with ingredients were stirred followed by a pasteurization step (90° C. for 20 minutes) to avoid contamination. Afterwards, each base was divided in 3 parts and each one inoculated with lactic acid bacteria (FDVS-YF-L01; Chr. Hansen Denmark, containing Streptococcus thermophilus) and three different yeasts, Pichia kluyveri strain 1, Pichia kluyveri strain 2 and Debaryomyces hansenii (kefir yeast) respectively, as shown in Table 8. The inoculation level of lactic acid bacteria was equal to 0.02% (wt), whereas the inoculation level of the yeast was 1×10⁶ cfu/g. In this example, fermentation was performed at 30° C. in 5 L scale. After the pH reached 4.55 the coagulum was broken with a perforated disk. Then, a cooling step to 12° C. followed by a mechanical post-treatment (2 bars back-pressure; FH Scandinox A/S, Tarm, Denmark) was performed to allow the smoothing process. The samples were collected in 120 ml plastic cups and stored at 6° C. for further analysis.

TABLE 8
Microorganisms used for fermentation of soy, coconut
an oat milk bases

| | Streptococcus | |
|---|---|---|
| | thermophilus | |
| Sample | (wt %) | Yeast |
| Soy 27 | 0.02% | Pichia kluyveri strain 1 |
| Soy 28 | 0.02% | Pichia kluyveri strain 2 |
| Soy 29 | 0.02% | Debaryomyces hansenii |
| Coconut 1 | 0.02% | Pichia kluyveri strain 1 |
| Coconut 2 | 0.02% | Pichia kluyveri strain 2 |
| Coconut 3 | 0.02% | Debaryomyces hansenii |
| Oat 1 | 0.02% | Pichia kluyveri strain 1 |
| Oat 2 | 0.02% | Pichia kluyveri strain 2 |
| Oat 3 | 0.02% | Debaryomyces hansenii |

Fermentation Profile

Fermentation profile was recorded with an Axone system (InLab® Sensors, Mettler Toledo).

Informal Sensory Evaluation

A sensory evaluation was performed on all the samples after 7 days from production. The assessors were asked to describe the samples.

Volatile Organic Compounds (VOCs) Analysis

VOCs were analyzed according the method described in Example 1.

Results

Fermentation Profile

FIG. 7 shows the time required for the pH reach 4.55 for all samples. No differences were observed between the tested yeasts. The pH could be reached rapidly in the coconut base followed by soy based and oat base.

Sensory Evaluation

Soy 27 (containing Pichia kluyveri strain 1) was described as very pleasant and fruity, with flavor resembling banana and pear. Soy 28 (containing Pichia kluyveri strain 2) was also described as pleasant and fruity similar to Soy 27 but less intense. In the fermented coconut (Coconut 1 and 2), the flavor contribution from Pichia kluyveri was considered aromatic and more intense than the fermented oat samples (Oat 1 and Oat 2). None of the above attributes were identified in samples where the Debaryomyces hansenii was used (Soy 3, Coconut 3 and Oat 3). The flavor of these samples containing the kefir yeast resembled beer or bread.

Volatile Organic Compounds (VOCs) Analysis

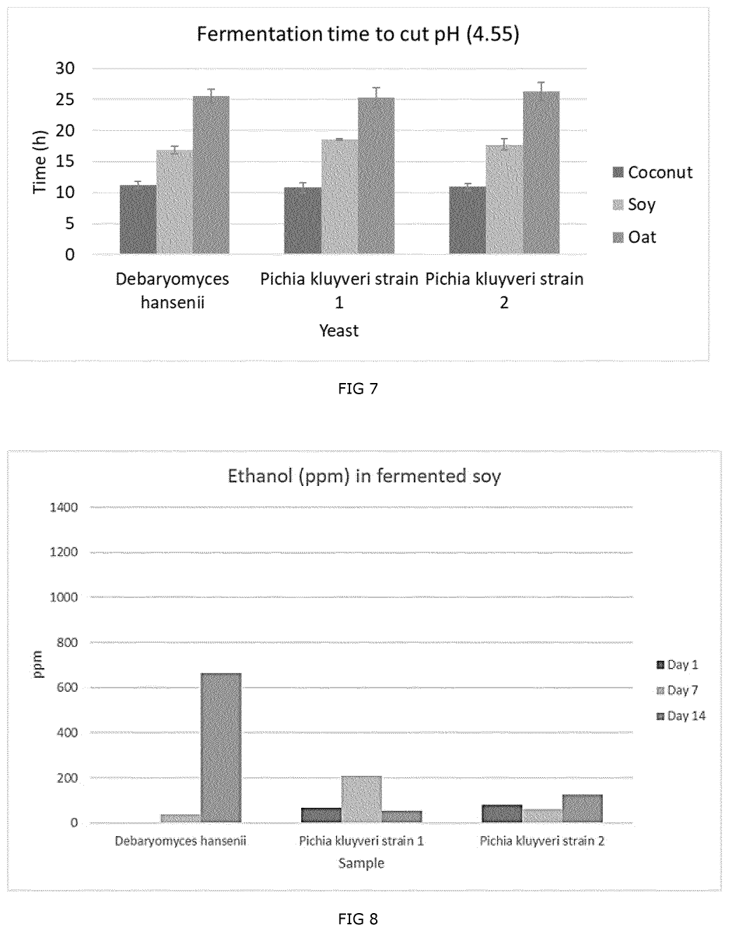

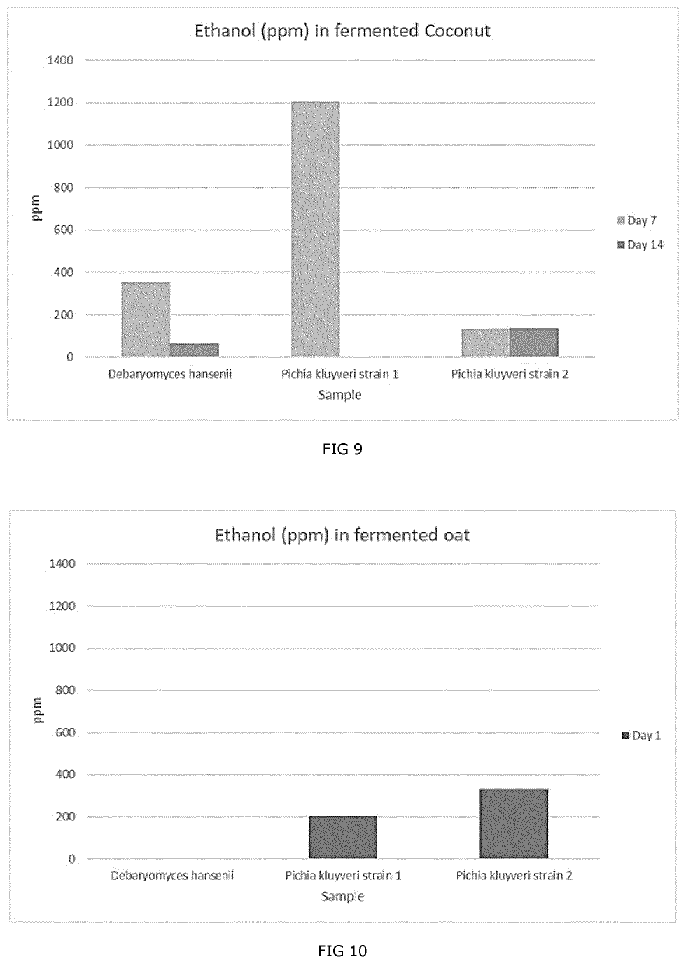

FIG. 8-10 shows the level of ethanol in milk bases prepared from soy, coconut and oat, respectively. Here, only very little ethanol was detected in all samples. Highest amount can be seen for coconut 1 at day 1 (about 1200 ppm) but was no longer detectable at day 14 likely due to assimilation by yeast.

### Example 5

Sample Preparation

Fresh milk from French supplier (Grignon farm) was standardized with tap water to contain 3.0% protein and pasteurized at 92° C. for 5 minutes.

After heat treatment, selected 3% carbohydrates were added to the milk as shown in Table 9, mixed and stored at 6° C. for 24 hours before use.

Milk base was warmed to 25° C. for 20 minutes and inoculated with lactic acid bacteria and yeast (Pichia kluyveri or Debaryomyces hansenii) at a level of around 10⁴ CFU/g (see Table 9). As lactic acid bacteria, F-DVS XPL-1 culture (Chr. Hansen A/S, Denmark) for mesophilic dairy products was used, which contains Streptococcus thermophilus, Lactococcus lactis subsp. cremoris, Lactococcus lactis subsp. lactis biovar. diacetylactis, Lactococcus lactis subsp. lactis, and Leuconostoc).

Fermentation of the milk bases was carried out at 25° C. until the pH reached 4.50. Afterwards, the samples were stored in cold chamber for further analysis.

TABLE 9

| | Carbohydrate | LAB starter culture | |
|---|---|---|---|
| Sample | (wt %) | (wt %) | Yeast |
| Kefir 1 | 3% glucose | 0.01% XPL-1 | 2.73 × 10⁴ Debaryomyces |
| | | (containing | hansenii |
| Kefir 2 | 3% glucose | Streptococcus | 1.75 × 10⁴ Pichia kluyveri |
| | | thermophilus, | strain 1 |
| Kefir 3 | 3% fructose | Lactococcus lactis | 2.73 × 10⁴ Debaryomyces |
| | | subsp. cremoris, | hansenii |
| Kefir 4 | 3% fructose | Lactococcus lactis | 1.75 × 10⁴ Pichia kluyveri |
| | | subsp. lactis biovar | strain 1 |
| Kefir 5 | 3% sucrose | diacetylactis, | 2.73 × 10⁴ Debaryomyces |
| | | Lactococcus lactis | hansenii |
| Kefir 6 | 3% sucrose | subsp. lactis, | 1.75 × 10⁴ Pichia kluyveri |
| | | Leuconostoc | strain 1 |

Yeast Enumeration

Samples stored at 6° C. were checked for yeast cell count every week (D+1; D+7; D+15; D+28). Enumeration of the yeast cells was performed by sampling 5 gram of samples to prepare dilutions. After serial dilution to the relevant dilution, samples were poured on YGC plate and incubated for 5 days at 25° C. in aerobic condition. After incubation, colonies (appearing white creamy, small and round) were counted.

Results

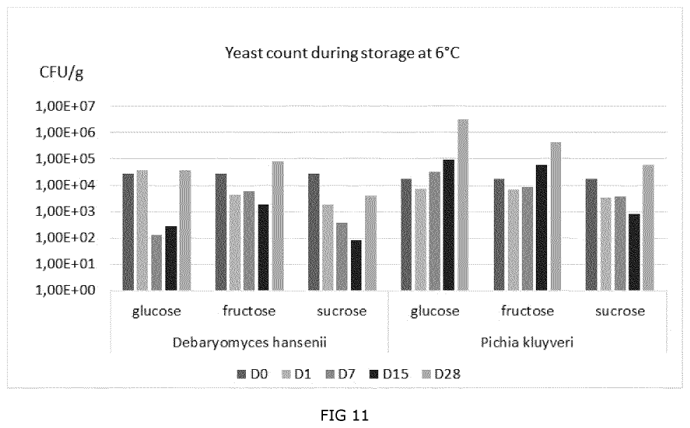

The yeast cell count of the samples is shown in FIG. 11. Compared to the commercial kefir yeast, Debaryomyces hansenii, cell count of Pichia kluyveri was generally higher in the period of 4 weeks. Sample with glucose (Kefir 2) had the highest cell count, followed by fructose (Kefir 4) and sucrose (Kefir 6).

Informal Sensory Evaluation

Samples prepared with Debaryomyces hansenii show typical taste of kefir with yeasty taste at the end shelf life. No specific flavor or smell could be detected at the start of the shelf life.

Samples prepared with Pichia kluyveri was described by one participant to have fruity smell and taste resembling cherry, peach or plum. Another participant described the flavor as fruity or ripen. Interestingly, the flavors were already detectable at the start of the shelf life and became more intense towards the end.

### Example 6

Preparation of Samples

Two samples were prepared the same way as Kefir 2 (with 3% glucose) and Kefir 4 (with 4% fructose) described in Example 5. Two further samples were also prepared the same way but fermented at a temperature (30° C.).

Ethanol Level

At day 14, 1 g of each four samples were measured for its ethanol content by gas chromatography.

Table 10 shows the ethanol level in 4 different kefir samples fermented with LAB starter culture and Pichia kluyveri at day 14.

TABLE 10

| Samples | Ethanol level |
|---|---|
| Kefir with 3% glucose | 275.52 ppm |
| Fermentation at 25° C. | |
| Kefir with 3% fructose | 376.99 ppm |
| Fermentation at 25° C. | |
| Kefir with 3% glucose | 310.6 ppm |
| Fermentation at 30° C. | |
| Kefir with 3% fructose | 274.2 ppm |
| Fermentation at 30° C. | |

This shows that low ethanol production in the range of 200 to 300 ppm can be achieved with the present invention.

### Example 7

Sample Preparation

The overall experimental design is summarized in Table 11. Organic unsweetened soy milk (NATURLI′ Foods A/S, Denmark) was used as starting material, and further blended with either no sugar, or different amount of glucose (Cargill), sucrose (Nordic sugar) and fructose (Hamburg Fructose GmbH International) with or without lactic acid (80%, S.Sørensen/Jungbunzlauer). In addition, some samples were inoculated with different lactic acid bacteria (FDVS-YF-L01, containing Streptococcus thermophilus, Chr. Hansen A/S, Denmark; and Lactobacillus paracasei, L. CASEI 431®, Chr. Hansen A/S, Denmark) as shown in Table 11. Soymilk was pasteurized (90° C. for 20 minutes) after adding sugars and/or lactic acid to avoid contamination. Pichia kluyveri strain 1 was inoculated in the amount 4 U/1000 L, or 1×10⁵ cfu/g and the lactic acid bacteria was inoculated as in the amount of 0.02% at the start of fermentation. In this example, fermentation was performed in 200 ml scale at 30° C. Fermentation was stopped either when the pH reached 4.55, or after 30 h maximum. After that, the coagulum was broken with a perforated disk and the samples were stored at 6° C. for further analysis. All analyses were performed after 10 days of storage at refrigerated conditions.

TABLE 11
Experimental design of Example 7

| Sample | Pichia | S. | L. | Sucrose | Fructose | Glucose |
|---|---|---|---|---|---|---|
| name | kluyveri | thermophilus | paracasei | 5%(wt %) | 3%(wt %) | 7.2%(wt %) |
| Soy 30 | X | X | | X | | |
| Soy 31 | X | X | | | X | |
| Soy 32 | X | X | | | | X |
| Soy 33 | X | | | | | X |
| Soy 34 | | X | | | | X |
| Soy 35 | | | X | | | X |
| Soy 36 | X | | X | | | X |

Pichia kluyveri Enumeration

Enumeration of Pichia kluyveri was performed as described in Example 1.

Volatile Organic Compounds (VOCs) Analysis

General Screening

For the screening of the volatile profile, VOCs were measured according to the method described in Example 1.

Specific Compound Quantification

For the quantification of the volatile organic compounds (VOCs), samples were analyzed according the method described in Example 1 by using the HS-GC-FID. Samples were prepared differently, by adding 1-2 g of NaCl and 600 μL of 0.1M NaF (pH=7) to 1 mL fermented sample and immediately analyzed by HSGC. This sample preparation allows the quantification of isoamyl acetate, which is a compound providing fruity, banana or pea-like flavor.

Results

Growth of Pichia kluyveri

Pichia kluyveri growth was evaluated in the samples after 10 days of storage, Pichia kluyveri was able to grow in all soy samples (data not shown).

General screen for volatile organic compounds (VOCs) The total amounts of esters (expressed as Signal to noise) measured in each sample are shown in Table 12. The inventor has surprisingly observed that the amount of total esters measured is higher in the samples combining Pichia kluyveri and LAB (Soy 32 and Soy 36), compared to Pichia kluyveri alone (Soy 33), YF-L01 (Streptococcus thermophilus) alone (Soy 34) or Lactobacillus paracasei, L. CASEI 431® alone (Soy 35). Especially high number of esters is found in the combination Pichia kluyveri and Streptococcus thermophilus (Soy 32).

TABLE 12
Total amounts of esters signal to noise ratio (S/N)

| | Sum of |
|---|---|
| Sample | esters (S/N) |
| Soy 33, Pichia kluyveri and glucose | 3357 |
| Soy 34, S. thermophilus and glucose | 1072 |
| Soy 35, L. paracasei and glucose | 663 |
| Soy 32, Pichia kluyveri, | 21449 |
| S. thermophilus and glucose | |
| Soy 36, Pichia kluyveri, L. paracasei | 7831 |
| and glucose | |

The specific amounts of isoamyl acetate in samples with Pichia kluyveri, YF-L01 (Streptococcus thermophilus) and added glucose (Soy 32), added fructose (Soy 31), and added sucrose (Soy 30) are shown in Table 13.

TABLE 13
Isoamyl acetate amounts in samples with added glucose

| | Isoamyl |
|---|---|
| | acetate |
| Sample | (ppm) |
| Soy 32, Pichia kluyveri, S. thermophilus and glucose | 5.7 |
| Soy 31, Pichia kluyveri, S. thermophilus and fructose | 3.1 |
| Soy 30, Pichia kluyveri, S. thermophilus and sucrose | 0.8 |

Isoamyl acetate is present in all samples, and especially for the sample with glucose.

1. A method for producing a fermented food product, comprising:
providing a milk base as a substrate,
adding at least one Pichia kluyveri strain and at least one lactic acid bacteria strain to the substrate,
fermenting the substrate in the presence of one or more carbohydrate(s) selected from sucrose, fructose and glucose, and
obtaining the fermented food product.

2. The method according to claim 1, wherein the method is for producing a fermented dairy analogue product, wherein the milk base is a plant milk base.

3. The method according to claim 1, wherein the method is for producing a fermented dairy product, wherein the milk base is an animal milk base.

4. The method according to claim 1, wherein the fermenting step is carried out in the presence of one or both of sucrose and fructose.

5. The method according to claim 1, wherein the at least one lactic acid bacteria strain comprises a homofermentative lactic acid bacteria strain.

6. The method according to claim 5, wherein the fermenting step is carried out in the presence of glucose.

7. The method according to claim 1, wherein the at least one lactic acid bacteria strain comprises a strain that ferments sucrose and releases fructose.

8. The method according to claim 1, wherein the fermenting step is carried out for 12 hours or longer.

9. The method according to claim 1, wherein the fermenting step is carried out until a pH below 4.6 is reached.

10. The method according to claim 2, wherein the plant milk base is selected from
a legume milk base prepared from soybeans, peas, beans, lupins, or lentils;
a nut milk base prepared from coconut, almond, cashew or walnut; and
a cereal milk base prepared from oat, wheat, rye or millet.

11. The method according to claim 1, wherein the milk base is a solution or suspension comprising whey protein(s), casein protein(s), or mixtures thereof.

12. The method according to claim 3, wherein the fermented dairy product is a kefir product comprising at least 10⁴ Colony Forming Units per gram (cfu/g) of Pichia kluyveri.

13. The method according to claim 1, wherein the Pichia kluyveri is one or more selected from:
(a) Pichia kluyveri deposited under accession number DSM 28484 at the German Collection of Microorganisms and Cell Cultures (Deutsche Sammlung von Mikroorganismen and Zellkulturen GmbH; DSMZ), Inhoffenstr. 7B, D-38124 Braunschweig, Germany;
Pichia kluyveri deposited under accession number V06/022711 at the National Measurement Institute, 541-65 Clarke Street, South Melbourne, Victoria 3205, Australia;
(c) Pichia kluyveri deposited under accession number V06/022712 at the National Measurement Institute, 541-65 Clarke Street, South Melbourne, Victoria 3205, Australia and
(d) mutants of any of deposited strains (a), (b) or (c).

14. A starter culture composition comprising a Pichia kluyveri strain and at least one homofermentative lactic acid bacteria strain selected from Streptococcus spp., Pediococcus spp., and Lactobacillus spp.

15. The starter culture composition of claim 14, further comprising one or more carbohydrates selected from sucrose, glucose and fructose.

16. The starter culture composition according to claim 14, wherein the at least one homofermentative lactic acid bacteria strain comprises a strain selected from Streptococcus thermophilus, Pediococcus acidilactici, and Lactobacillus plantarum.

17-18. (canceled)

19. The method according to claim 1, wherein the at least one lactic acid bacteria strain comprises a homofermentative strain of Streptococcus thermophilus.

20. The method according to claim 1, wherein the fermenting step is carried out for a period of time selected from 12 hours, 18 hours, 24 hours, 36 hours, and 48 hours.

21. The method according to claim 1, wherein the fermenting step is carried out until a pH of 4.55 is reached.
